(19) United States
(12) Patent Application Publication
(10) Pub. No.: US 2003/0035794 A1
(43) Pub. Date: Feb. 20, 2003

(54) GENE-THERAPEUTIC NUCLEIC ACID CONSTRUCT, PRODUCTION OF SAME AND USE OF SAME IN THE TREATMENT OF HEART DISORDERS

(75) Inventors: WOLFGANG-M. FRANZ, GROSS GRONAU (DE); THOMAS ROTHMANN, LANGENFELD (DE); H.A. KATUS, RATZEBURG (DE)

Correspondence Address: BIRCH STEWART KOLASCH & BIRCH, PO BOX 747, FALLS CHURCH, VA, 22040-0747, US

(73) Assignee: BIRCH, STEWART, KOLASCH & BIRCH, LLP

(21) Appl. No.: 09/068,751

(22) PCT Filed: Nov. 14, 1996
(86) PCT No.: PCT/DE96/02181

(30) Foreign Application Priority Data
Nov. 17, 1995 (DE) 195 42 838.2
Oct. 1, 1996 (DE) 196 40 630.7

(51) Int. Cl. A61K 48/00

(52) U.S. Cl. 424/93.21; 514/44

(57) ABSTRACT

The invention pertains to a gene therapeutic nucleic acid working model containing a regulatory nucleic acid sequence of the 5′ end myosin light chain 2 gene (MLC 2) of the heart that is functionally connected to a nucleic acid, which is encoded for a therapeutically effective gene product, an antisense nucleic acid, or a ribosome, as well as a process for its production and application for the gene therapeutic treatment of heart disease.

The invention concerns a gene therapeutic nucleic acid working model containing a regulatory nucleic acid sequence of the 5′ end of the myosin light chain 2 gene (MLC 2) of the heart connected functionally to a nucleic acid, which is encoded for a therapeutically effective gene product, for an antisense nucleic acid, or for a ribosome, as well as a process for its production and its use in gene therapeutic treatment of heart disease.

The syndrome of cardiomyopathy comprises a group of heart muscle disorders that become manifest as contractile as well as electrophysiologic disorders, and finally lead to severe heart insufficiency and/or sudden electrophysiologic heart death. The search for monogenetic causes in the familiar forms of dilative and hypertrophic cardiomyopathies is, at this time, the object of numerous scientific investigations. The causes for heart muscle disease at the molecular level were discovered just recently. For example, so-called Duchenne muscle dystrophy (DMD) also causes cardiomyopathy. DMD is a hereditary disease caused by mutations and deletions in the dystrophic gene. The dystrophic gene is located on the X chromosome and is expressed in healthy human beings, for example, in the heart muscle cells. It was also found that, in chronic congestive heart failure (CHF), the myocardium contains 50% less β-adrenergic receptor than the healthy myocardium.

After identifying genetic defects or evidence of a modified gene expression in diseased heart muscle tissue, there is a possibility of curing the disease by means of molecular biologic methods. In this way, for example, the somatic gene transfer represents a very promising method for treating genetically caused muscle disease.

Different methods such as, for example, gene transfer by injection of DNA, liposome-supported gene transfer, or gene transfer by means of retroviral, adenoviral, or adeno-associated vectors are suitable for the somatic transfer. Essential requirements for a successful gene therapy are a high transfer rate, a stable gene expression and, above all, tissue specificity.

Successful gene transfer and the successful expression of a gene encoded for β-galactosidase under the control of CMV promoters, as well as in smooth muscle cells of the coronary vessels and in heart muscle cells, are shown in WO94/11506. However, a heart muscle-specific expression could not be obtained. In the description there is reference generally to the heart muscle-specific troponin C (cTNC) promoter, but without showing a heart-specific in vivo expression.

From Franz, W. -M. et al (1994), Cardioscience (5, 235-243, No. 4), we learn that the microinjection of a naked DNA of myosin light chain 2 (MLC 2) promoter luciferase fusion gene into a male pronucleus of fertilized mice oocytes causes a transgenic mouse that possesses a heart muscle-specific expression of the luciferase.

Myosin, a main component of the heart muscle and other striped muscles, consists of two heavy chains (MHC) and two pairs of myosin light chains (MLC). The MLC are divided again into non-phosphorizable (MLC 1) and phosphorizable (MLC 2) forms. It was found now that the regulatory nucleic acid sequence (promoter) is differentiated at the 5′ end of the MLC 2 gene of the skeletal muscle and the heart muscle of rats, but that the MLC 2 gene of the heart muscle of rats and chickens is preserved, even though rats and chickens are separated from an evolutionary point of view (Henderson, S. A. et al. (1989), J. Biol. Chem., 264, 18142-18148). Lee et al. (Lee, K. J. et al. (1994), Mol. Cell. Biol., 14, 1220-1229, No. 2) found, with respect to transgenic rats, that a combination of positive (HF 1a and HF 1b) and negative (E box and HF 3) regulatory elements that lie within 250 base pairs upstream of the transcription starting point, cause a ventricle chamber-specific expression, even though the receipt of the specificity in a gene therapeutic in vivo application could not be demonstrated until now. However, Franz, W. -M. et al. (1994) cited above found that, also based on transgenic rats, a further regulatory sequence, the so-called heart-specific sequence (CSS), a repressor element lying approx. 1700 base pairs upstream of the transcription starting point, is necessary for the heart muscle-specific expression. From these results, it can be recognized that the mechanism for the heart-specific expression of genes has still not been explained and a heart-specific expression of a gene after in vivo application of the gene has not yet been found.

The object of the invention is therefore to find a nucleic acid working model that possesses a high transfer rate, a stable gene expression and, above all, a specificity for heart muscle cells for gene therapy of heart disease.

One object of the invention is, therefore, a gene therapeutic nucleic acid working model containing a regulatory nucleic acid sequence of the 5′ end of myosin light chain 2 gene (MLC 2) of the heart, preferably the heart of a mammal, particularly of human beings or rodents, particularly rats, which are functionally connected to a nucleic acid and encoded as a therapeutically effective gene product, for an antisense nucleic acid, or a ribosome.

The regulatory nucleic acid sequence, in the sense of the invention, is understood generally to mean the nucleic acid sequence lying upstream of the transcription starting point (+1) of the MLC 2 gene that controls the transcription of the nucleic acid sequence connected to this sequence at the 3′ end lying upstream, particularly with respect to the correct transcription starting point, the transcription rate and/or the heart muscle tissue specificity, that is, the regulatory nucleic acid sequence is functionally connected to the upstream lying nucleic acid sequence. The sequence from approximately +18 to −19 up to approximately −800 with respect to the transcription starting point of MLC 2 gene of the heart is particularly preferred (see FIG. 10), since it was particularly surprising that approximately 800 base pairs upstream of the transcription starting point were sufficient to effect a heart-specific, and particularly a heart chamber-specific, expression in an in vivo application, even though this sequence does not contain the so-called heart-specific sequence CSS. A further preferred embodiment is also a sequence from approximately +18 to −19 up to approximately −1600, and particularly from approximately +18 to −19 up to approximately −1800, above all from approximately +18 to −19 up to approximately −2100 or from approximately +18 to −19 up to approximately −2700 with respect to the transcription starting point of the MLC 2 gene of the heart (see FIG. 10). The regulatory nucleic acid sequence contains, above all, one or several regulatory elements selected as TATA box, HF 1a, HF 1b, HF 2, HF 3, E box, MLE1 and/or CSS sequence, particularly selected as TATA box, HF 1a, HF 1b, HF 2, HF 3, E box and/or MLE1. For example, in a regulatory nucleic acid sequence of rats, the TATA box lies approximately between −198 and −19, the HF 1 element, a preserved 28-base long sequence, approximately between −72 and −45, and particularly the HF 1a element approximately between −57 and −65 and the HF 1b element approximately between −45 and −57 and −65, and in that HF 1b element lies approximately between −45 and −56, the HF 2 element approximately between −123 and −134, the HF 3 element approximately between −186 and −198, the E box element approximately between −72 and −77, the MLE1 element approximately between −165 and −176, and the CSS-like element approximately between −1723 and −1686 with respect to the transcription starting point of the MLC 2 gene (see FIG. 10). The regulatory sequences TATA box, HF 1b element, HF 1a element, E box element, HF 2 element, MLE1element and HF 3 element lie in the MLC 2 gene of rats in this order within the first 200 bases upstream of the transcription starting point of the gene (see FIG. 10).

For the heart-specific expression, it is preferable that the nucleic acid working model according to the invention contain the HF 1a element, the HF 1b element, the MLE1 element and the HF 3 element, preferably together with the E box, particularly together with the E box element and/or HF 2 element. In any case, it is also preferable the nucleic acid working model of the invention contain additionally the heart specific sequence CSS.

Under a gene therapeutic nucleic acid working model in the sense of the invention is understood as a nucleic acid working model with a nucleic acid sequence that is particularly a DNA or RNA sequence, preferably one with a single or double strand, above all a double strand DNA sequence, whereby the nucleic acid working model can be used for treating heart insufficiency, dilative or hypertrophic cardiomyopathies, dystrophinopathy, vessel disorders, high blood pressure, arteriosclerosis, stenosis or restenosis of the blood vessels in an advantageous manner.

The nucleic acid working model of the invention is preferably combined with a virus vector and/or with liposomes, preferably ligated with an adenovirus, above all with a replication-deficient adenovirus vector, or with an adeno-associated virus vector, above all with an adeno-associated virus vector that consists exclusively of two inverted terminal repeat sequences (ITR). A particularly preferred embodiment of the invention is the gene technical connection of the nucleic acid working model of the invention with an adenovirus vector, above all with a replication-deficient adenovirus vector.

An adenovirus vector, and particularly a replication-deficient adenovirus vector, is preferred for the following reasons:

The human adenovirus belongs to the class of double strand DNA viruses with a genome of approximately 36 kilobase pairs (Kb). The viral DNA encodes for approximately 2700 different gene products, wherein early (“early genes”) and late (“late genes”) gene products are differentiated with respect to the adenoviral replication cycle. The “early genes” are divided into four transcriptional units E1 to E4. The late gene products encode for the capsid protein. Immunologically, they can identify at least 42 different adenoviruses and the subgroups A-F, which are suitable for the invention. The transcription of the viral gene presupposes the expression of the E1 region, which is encoded for a transactivator of the adenoviral gene expression. This dependency of the expression of all following viral genes from the E1 transactivator can be used for the construction of the non-replicable adenoviral vectors (see for example B. McGrory, W. J. et al. (1988) Virol. 163, 614-617 and Gluzman, Y. et al. (1982) in “Eukaryotic Viral Vectors” (Gluzman, Y., ed.) 187-192, Cold Spring Harbor Press, Cold Spring Harbor, N.Y.). In adenoviral vectors, particularly of type 5 (for sequence see Chroboczek, J. et al. (1992) Virol. 186 280-285) and, above all, of the subgroup C, generally the E1 gene region is substituted by a foreign gene with its own promoter or by means of the nucleic acid working model of the invention. By means of the exchange of the E1 gene region, which is a prerequisite for the expression connected with adenoviral genes, there results a non-replicable adenovirus. These viruses can only then multiply in one cell line that substitutes the missing E1 gene.

Replication-deficient adenoviruses are generally formed therefore by homologue recombination in the so-called 293 lines cell line (human embryo kidney cell line), which has a stable copy of the E1 region integrated into the genome. For this purpose, a nucleic acid sequence (for example, for a therapeutically effective gene product or for a marker, for example β-galactosidase/β-gal) under control of its own promoter (for example, of the mlc 2 promoter according to the invention) is cloned in recombinated adenoviral plasmids. The homologue recombination takes place also, for example, between the plasmids pAd.mlc 2/β-gal and an E1-deficient adenoviral genomes such as, for example d1327 or de1324 (adenovirus 5) in the helper cell line 293. If the recombination is successful, the viral plaques are gathered. The replication-deficient viruses generated in this way are then used in high titers (for example 10⁹ to 10¹¹ “plaque forming units” or [plaque forming units]) for infection of the cell culture or for the somatic gene therapy.

Generally, the exact insertion location of the foreign DNA into the adenoviral genome is not critical. It is also possible, for example, to clone the foreign DNA at the location of the deleted E3 gene (Karlsson, S. et al. EMBO J. 1986, 5, 2377-2385). Preferably, however, the E1 region or parts thereof, for example, the E1A or E1B region (see for example WO95/00655) are substituted by the foreign DNA, above all if also the E3 region is deleted.

However, the invention is not limited to the adenoviral vector system, but also adeno-associated virus vectors are suitable in combination with the nucleic acid working model according to the invention due to the following reasons in particular:

The AAV virus belongs to the family of parvoviruses. These are characterized by an ikosaedric unprotected capsid with a diameter of 18-30 nm, which contains a linear single-strand DNA of approximately 5 Kb. A coinfection of the host cell with helper viruses is necessary for an efficient multiplication of the AAV. As helpers are suitable, for example, adenoviruses (Ad5 or Ad2), herpes viruses and vaccinia viruses (Muzyczka, N. (1992) Curr. Top. Microbiol. Immunolog. 158, 97-129). In the absence of these helper viruses, the AAV passes into a latent state, wherein the virus genome is able to integrate itself in a stable manner into the host virus. The capability of the AAV to integrate into the host genome makes it particularly interesting as a transduction vector for mammal cells. Generally, both approximately 145 bp long inverted terminal repeat sequences (ITR: inverted terminal repeats; see for example WO95/23867) are sufficient for the vector function. They carry the signals for replication, packaging and integration necessary in “cis” in the host cell genome. A vector plasmid, which carries the genes for non-structural protein (rep protein) and for structural protein (cap protein), for packaging into recombinated vector particles is transmitted into the adenovirus-infected cells. A cell-free lysate is produced after a few days, which contains adenoviruses aside from the recombinated AAV particles. The adenoviruses can also be removed advantageously by means of heating to 56° C. or by banding in the cesium chloride gradient. With this cotransfection method, the rAAV titer of 10⁵-10⁶ IE/ml can be obtained. The contamination by means of wild-type viruses lies below the evidence limit if the packaging plasmid and the vector plasmid do not have overlapping sequences (Samulski, R. J. (1989) J. Virol. 63, 3822-3828).

The transfer of foreign genes in somatic body cells can be carried out by means of AAV in at-rest differentiated cells, which is particularly advantageous for gene therapy of the heart. A long-lasting gene expression in vivo can be obtained by means of the mentioned integration capability, which is again particularly advantageous. Another advantage of AAV is that the virus is not pathogenic for humans and is relatively stable in vivo. The cloning of the nucleic acid working model of the invention in the AAV vector or parts thereof takes place according to the methods known to the experts, such as, for example, the ones described in WO95/23867 by Chiorini, J. A. et al (1995) Human Gene Therapy 6, 1531-1541 or Kotin, R. M. (1994) Human Gene Therapy 5, 793-801.

Another advantageous combination in the sense of the invention is the complexing of the nucleic acid working model according to the invention with liposomes, since in this way a very high transfection efficiency, particularly of heart muscle cells, can be obtained (Felgner, P. L. et al. (1987) Proc. Natl. Acad. Sci. USA 84, 7413-7417). In the lipoinfection, small unilamellar vesicles of cationic lipids are produced by means of ultrasound treatment of the liposome suspension. The DNA is ionically bonded on the surface of the liposomes at such a rate that a positive net load remains and the plasmid DNA is complexed to 100% from the liposomes. Aside from the lipid mixtures (used by Felgner et al. (1987, see above) DOTMA (1,2-dioleyloxypropyl-3-trimethyl-ammonia bromide) and DOPE (dioleylphosphatidylethanolamine), also numerous new lipid formulations have been synthesized since then and have been tested as to their efficiency in the transfection of different cell lines (Behr, J. P. et al. (1989) Proc. Natl. Acad. Sci USA 86, 6982-6986; Felgner, J. H. et al. (1994) J. Biol. Chem. 269, 2550-2561; Gao, X. & Huang, L. (1991) Biochem. Biophys. Res. Commun. 179, 280-285; Zhou, X. & Huang, L. (1994) Biochem. Biophys. Acta 1189, 195-203). Examples of the new lipid formulations are DOTAP N-(1,3-dioleoyloxy)propyl)-N,N,N-trimethylammonia methylsulphate or DOGS (TRANSFECTAM; dioctadecylamidoglycylspermin). An example for the production of DNA liposome complexes and their successful use in the heart specific transfection is described in DE 4,411,402.

Suitable therapeutic gene products include, for example, dystrophin, the β-adrenergic receptor, nitrogen monoxide synthase or any other products which, for example, complement a monogenetic fault, impede or reduce electrophysiologic disturbances, or diminish the severity of or cure heart specific diseases. It is particularly advantageous if the gene that is encoded for the therapeutic gene product (transgene) contains one or several non-encoded sequences, including intron sequences, preferably between promoter and startcodon of the transgene, and/or a polyA sequence at the 3′ end of the transgene, for example, a SV40 virus polyA sequence, since in this way a stabilization of the mRNA of the heart muscle cell can be achieved (Jackson, R. J. (1993) Cell 74, 9-14 and Palmiter, R. D. et al. (1991) Proc. Natl. Acad. Sci. USA 88, 478-482).

The nucleic acid that is functionally bound with the regulatory nucleic acid of the MLC 2 gene, however, can be not only a nucleic acid that is encoded as a therapeutically effective gene therapy, but also a nucleic acid that is encoded for an “antisense” nucleic acid, preferably an “antisense” oligonucleotide, particularly an “antisense” DNA oligonucleotide, or for a ribosome. The expression of the gene in the heart can be specifically reduced or impeded by means of “antisense” oligonucleotides as also by means of ribosomes, whereby several heart specific diseases, such as for example atherosclerosis or restenosis, and also autoimmune or cancerous diseases, can be treated (see for example B. Barr, E. & Leiden, J. M. (1994) Trends Cardiovasc. Med. 4, 57-63, No. 2 and Bertrand, E. et al. (1994) Nucleic Acids Res. 22, 293-300).

Another object of the invention is also a process for producing the nucleic acid working model, whereby the regulatory nucleic acid sequence described more extensively above is connected functionally with a nucleic acid that is encoded for a therapeutically effective product, an antisense nucleic acid, or for a ribosome. In a preferred embodiment, the named regulatory nucleic acid and the nucleic acid that is encoded for a therapeutically effective gene product, an antiseme nucleic acid, or a ribosome, are cloned either simultaneously or one after the other in one of the virus vectors more extensively described above.

The process of the invention takes place according to the methods generally known to the experts (see, for example, Maniatis et al. (1982) Molecular Cloning, A Laboratory Manual, Cold Spring Harbor Laboratory, New York). The protein or nucleic acid sequences of the therapeutically effective gene products, for example, can be obtained by means of the EMBL gene bank or other gene banks available to the public. The sequence of the MLC 2 gene of the heart of rats is known from Henderson, S. A. et al. (1989), cited above, and the nucleic acid sequence of MLC 2 genes can be seen in FIG. 10. Starting from these sequences and the method of Henderson, S. A. et al. (1989) described above for isolating the MLC 2 gene, including the regulatory sequences of a genomic gene bank, sequences homologous to rat genes can also be found without difficulty in the genes of other animals or human beings. Specifically, it is possible to isolate other regulatory sequences of the MLC 2 gene of the heart in genomic gene banks of other animals or of humans without unexpected expense since, as was mentioned above, the regulatory nucleic acid sequences of the MLC 2 gene of the heart lying on the 5′ end are generally essentially maintained, even among the evolutionarily most distant kinds of animals, for example, rats and chickens (Henderson, S. A. et al. (1989), cited above).

Another object of the invention concerns a process wherein the nucleic acid working model is complexed with liposomes, as is described in more detail, for example, in DE 4,411,402.

Another object of the invention is also the use of the nucleic acid working model for gene therapeutical treatment of heart disorders or for producing a medication for gene therapeutical treatment of a heart disorder, wherein the heart disease is preferably a heart insufficiency, dilative or hypertrophic cardiomyopathy, dystrophinopathy, blood vessel disorder, high blood pressure, atherosclerosis, stenosis, and/or restenosis of the blood vessels. It is particularly advantageous if the nucleic acid working model of the invention is effective essentially in the heart chamber (ventricle).

Another object of the invention is therefore also a medication containing a nucleic acid working model according to the invention and, if necessary, a pharmaceutical carrier, which can be, for example, a physiologic puffer solution, preferably containing a pH from approximately 6.0 to approximately 8.0, from approximately 6.8 to approximately 7.8, approximately 7.4, and/or an osmolarity from approximately 200 to approximately 400 milliosmols per liter (mosm/l), preferably from approximately 290 to approximately 310 mosm/l. The pharmaceutical carrier can also contain suitable stabilizers, such as, for example, nuclease inhibitors, preferably complex builders such as EDTA, and/or other auxiliary substances known to the experts.

The application of the nucleic acid working model of the invention, if necessary in combination with the above-described virus vectors or liposomes, generally takes place intravenously (i.v.), for example, with the aid of a catheter. The direct infusion of the nucleic acid working model according to the invention, especially in the form of recombined adenoviruses, into the coronary arteries of the patient (“percutaneous coronary gene transfer,” PCGT), is advantageous. The application of the nucleic acid working model is especially preferred mainly in the form of recombinant adenoviruses with the aid of a balloon catheter, such as described, for example, in Feldman et al. (Feldman, L. J. et al. (1994) JACC 235A, 906-34), since in this way the transfection is limited not only to the heart, but can also be limited within the heart to the infection location.

The unexpected advantages of the invention are found in that the nucleic acid working model of the invention shows a high transfer rate in the gene therapeutic treatment of heart disease, wherein transfected cells are stable and expressible and, above all, they do not loose their specificity for the heart muscle cells. This is very surprising because, for example, the smmhc promoter loses its specificity for neonatal and adult smooth muscle cells (see Example 6 below) and a preferred mlc 2 promoter of the nucleic acid working model according to the invention, which does not contain the heart specific sequence CSS, conserves its specificity particularly in connection with an adenovirus vector. By specificity in the sense of the invention is understood, therefore, that expression that is controlled by the mlc 2 promoter in cardiomyocytes, particularly in the ventricle, and which is clearly higher than, for example, the expression controlled by the mlc 2 promoter in the vessel muscle cells wherein the difference in the expression amounts approximately from one to approximately three, particularly from approximately three to approximately six, above all, from approximately three to approximately four decimal power.

It was also surprising that the mlc 2 promoter limited the expression of luciferidase more to the heart than the αmhc promoter (see Example 10 below). A particular advantage is also that, with the nucleic acid working model according to the invention, the heart-specific expression after in vivo application is limited to the heart chamber (ventricle) (see Example 11 below), since in this way, for example, it is possible to increase the contraction force of the ventricle.

The following drawings and examples will explain the invention further without placing limitations on the same.

### DESCRIPTION OF THE DRAWINGS

FIG. 1 shows a schematic representation of the constructed plasmids pAd-Luc, pAd-rsvLuc, pAd-mlcLuc, and pAd-smmhcLuc. BamHI, KpnI and HindIII designate the restriction enzyme interfaces of the corresponding enzyme. ITR represents “inverted terminal repeat,” ψ represents the packaging sequence, mlc 2 represents the “myosin light chain”-2v promoter, luciferase represents the luciferase encoding sequence, Ad 9.4-18 m.u. represents the adenoviral sequence of 9.4 to 18 “map units” (1 m.u. - 360 bp) of adenovirus type 5 and ori/ampR represents the “origin of replication” and the ampicillin resistance gene.

FIG. 2 shows the recombined adenoviruses obtained by means of homologue recombination that come from the adenovirus de1324, wherein the luciferase gen is cloned in the former E1 region. The expression of the luciferase gen is controlled by either the smmhc promoter (Ad-smmhcLuc) that is specific for the smooth vessel musculature, the mlc-2v promoter (Ad-mlcLuc) expression specific for the heart muscle, the RSV promoter (Ad-rsvLuc) as positive control, or by means of no promoter (Ad-Luc) as negative control. The abbreviations are similar to the ones in FIG. 1.

FIGS. 3A-C show schematic representations of the luciferase activities of Ad-Luc, Ad-rsvLuc, Ad-mlcLuc, and Ad-smmhcLuc in different cell lines. The thin line in each column represents the average standard deviation.

FIGS. 4A-C show schematic representations of the luciferase activities of Ad-Luc, Ad-rsvLuc, and Ad-mlcLuc in different primary cell tissues. The thin line in each column represents the average standard deviation of the experiments.

FIGS. 5A-C show schematic representations of the luciferase activities of Ad-Luc, Ad-rsvLuc, and Ad-mlcLuc in different tissues after injection of recombined adenoviruses in the heart chamber of neonatal rats. The thin line in each column represents the average standard deviation of the experiments.

FIGS. 6A and B show the histologic evidence of the β-galactosidase activity in the myocardium after intracavitary injection of recombined adenovirus AD.RSVβgas. FIG. 6A represents a photograph of a histologic section through the apex (injection location). FIG. 6B represents the photograph of a histologic section through the left ventricle. The bar graph corresponds to 100 μm.

FIGS. 7A-C show the evidence of adenoviral DNA in 12 different tissues after intracavitary injection of the recombined adenoviruses Ad-Luc, Ad-rsvLuc, and Ad-mlcLuc.

FIG. 7A shows a photograph of a 2.4% agaro sail with the specific 860 bp PCR product, which was obtained by means of amplification from decreasing amounts of Adde1324 DNA. As sample were used 100 ng genomic DNA of rats mixed with Adde1324 DNA: trace 1: 10 pg; trace 2: 1 pg; trace 3: 100 fg; trace 4: 10 fg; trace 5: 1 fg; trace 6: 0.1 fg; trace 7: no viral DNA. M corresponds to a DNA marker (100 bp leader).

FIG. 7B shows a photograph of a 2.4% agaro sail with the specific 860 bp PCR product, amplified from 100 ng genomic DNA, which was isolated from the cited tissues after intracavitary injection of Ad-Luc, Ad-rsvLuc, and Ad-mlcLuc. A PCR base with 100 ng genomic DNA of rats mixed with 1 pg Adde1324 DNA served as positive control, a base without Adde1324 DNA served as negative control.

FIG. 7C shows a Southern-blot of Ad-mlcLuc of the infected animal according to FIG. 7B. The ^{32p}-marked 860 bp PCR product of a control base was used as probe.

FIGS. 8A and B show the luciferase activities of the recombined adenoviruses Ad-αmhcLuc (FIG. 8A) and Ad-mlcLuc (FIG. 8B) after intracavitary injection in the left main chamber of neonatal rats in different tissues.

FIGS. 9A-C show the luciferase activities of recombined adenoviruses Ad-rsvLuc, Ad-mlcLuc, Ad-αmhcLuc, and Ad-Luc in the atrium (FIG. 9A) and in the ventricle (FIG. 9B). The relationship between the activities in the atrium and in the ventricle is shown in FIG. 9C. The columns show the averages of four experiments, wherein the points represent the results for each tested animal or the relationship of the luciferase activity in the ventricle with respect to the atrium.

FIGS. 10A-C show the nucleic acid sequence of a 2216 base pair-long promoter of the MLC-2v gene of rats, lying upstream of the transcription starting point (+1). The nucleic acids of positions 1-156 encode for the packaging sequence ψ of adenovirus Ad5 (positions 300-456). The cloning sequence for the restriction endonuclease BamHI is located at position 158-163 and for KpnI at position 189-194. At position 189-2405 is located the 2216 base pair-long promoter of the MLC-2v gene. The CSS-like sequence is located at position 682-724, the HF element at position 2207-2219, the MLE1 element at position 2229-2241, the HF 2 element at position 2271-2289, the E box element at position 2328-2333, the HF 1a element at position 2340-2348, the HF 1b element at position 2349-2361 and the transcription start (+1) at position 2406. The luciferase encoding sequence starts at position 2461. At position 1660-2406 lies the 746 base pair-long regulatory sequence of the plasmid pAd-mlcLuc (see Example 1).

### EXAMPLES

### 1. Production of Recombinant Plasmids pAd-Luc, pAd-rsvLuc, pAd-mlcLuc, pAd-smmhcLuc, and pAdαmhcLuc

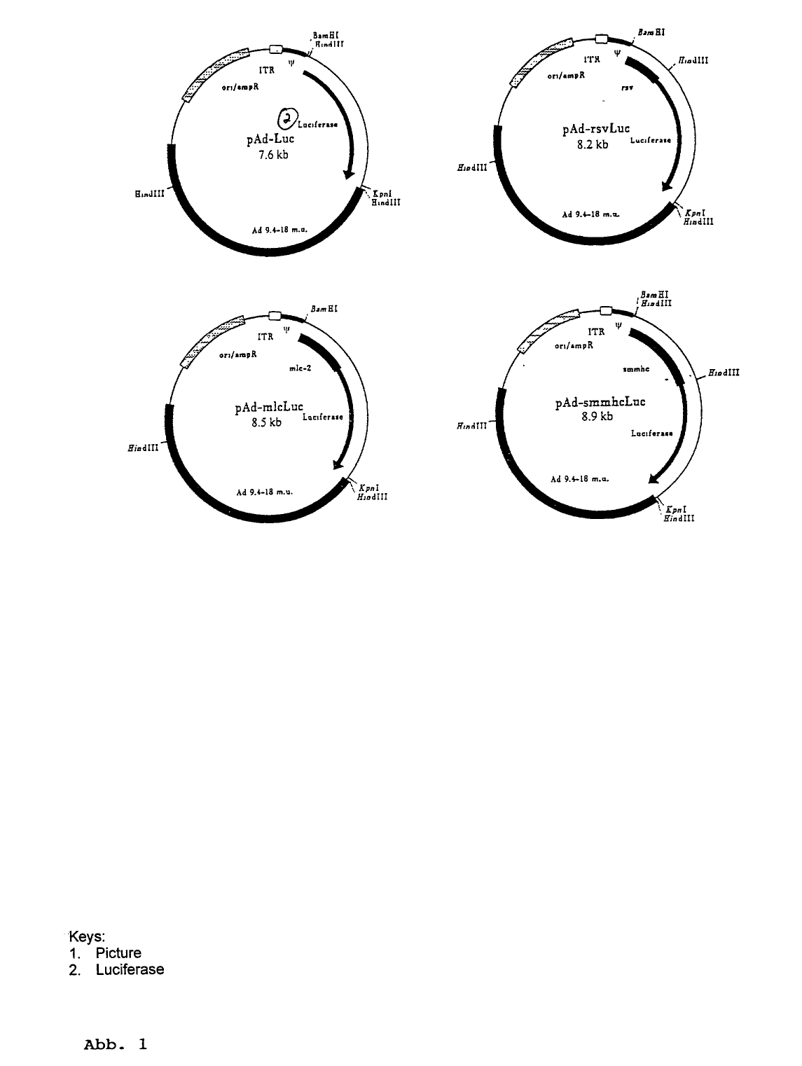

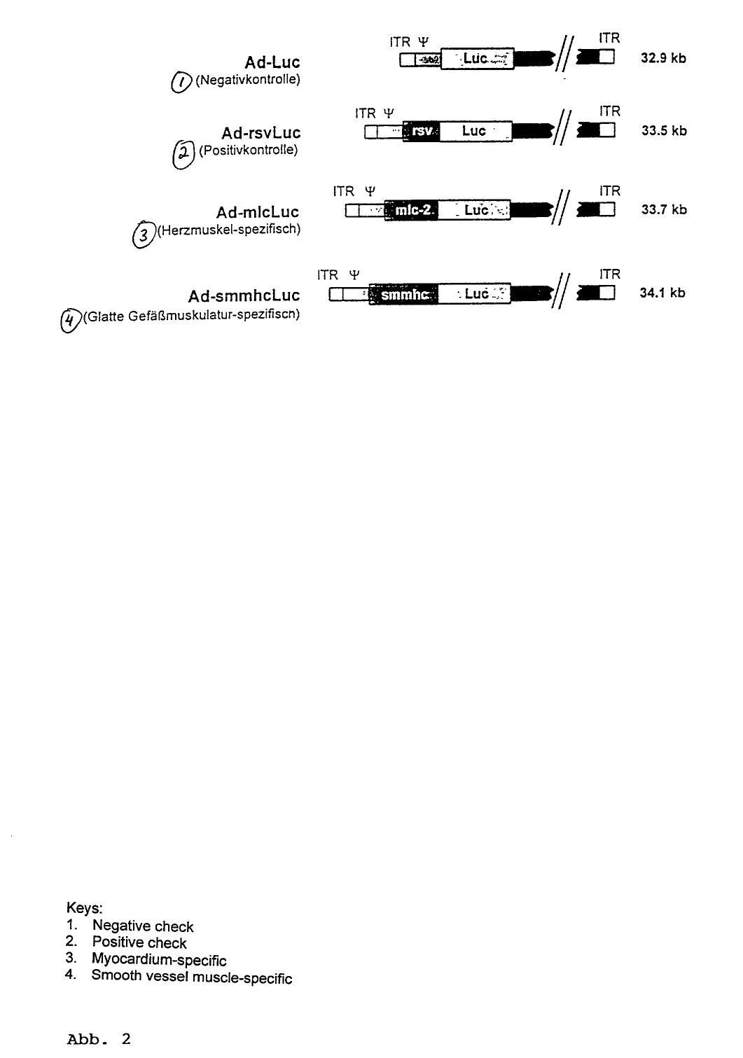

The plasmids pAd-Luc, pAd-rsvLuc, pAd-mlcLuc, pAd-smmhcLuc (FIG. 1) and pAdαmhcLuc are derivates of the plasmids pAd.RSVβgal (Stradtford-Perricaudet, L. D., J. (1992) Clin. Invest. 90, 626-630), wherein the BamHI-KpnI RSV-βgal cassette (“Rous sarcoma virus” promoter and β-galactosidase reporter gene) is exchanged against the luciferase cDNA with its endogenous polyadenylation signal for either one promoter (pAd-Luc), for the RSV promoter (pAD-RSV-Luc), the mlc-2v promoter (pAD-mlcLuc), the “smooth muscle myosin heavy chain” promoter (pAD-smmhcLuc), or the “α-myosin heavy chain” promoter (pAD-αmhcLuc). For this purpose, the HindIII/KpnI fragment of the plasmid pSVOAL, which is encoded for the luciferase gene, 5′ in the HindIII/KpnI cloning interfaces of the vector pBluescriptSK (strata gene) is subcloned and the plasmid pBluescript-Luc is generated thereby (Wet, J. R. et al. (1987) Mol. Cell. Biol. 7, 725-735). The BamHI/KpnI luciferase fragment of the subclone pBluescript-Luc was then cloned at the BamHI/KpnI interfaces of the plasmid pAD.RSV-βgal and the plasmid pAd-Luc was generated thereby.

For the cloning of the plasmid pAD-rsvLuc, the BamHI/HindIII RSV fragment (587 bp) of the plasmid pAD.RSV-βgal is cloned in the BamHI/HindIII interfaces of the subclone pBluescript-Luc and the plasmid pBluescript-RSV-Luc is generated thereby. The BamHI/KpnI RSV luciferase fragment of the plasmid pBluescript-RSV-Luc is then cloned in the BamHI/KpnI interfaces of pAD.RSV-βgal and the plasmid pAd-rsvLuc is generated thereby.

For producing the plasmid pAD-mlcLuc, the BamHI/KpnI mlc-luciferase fragment (746 base pair-long “myosin light chain”-2v promoter according to FIG. 10 and 1.8 kb luciferase gene) was cloned from the plasmid mPLCLΔ5′ directly into the BamHI/KpnI interfaces of the plasmid pAd-RSVβgal (Henderson, S. A. et al (1989) J. Biol. Chem. 264, 18142-18148). For this purpose, the mlc-2/luciferase fusion working model was cut out of the restriction enzyme interface KpnI, the overhanging ends in a so-called 37 Klenow reaction” were filled in, and PvuII left was ligated at both ends. The 4.0 kb long mlc-2/luciferase DNA fragment was then coupled similar to the recombined plasmid pAd.RSVβgal in the PvuII interface at the 3′ end of the 1.3 m.u. region of the adenovirus type 5 (Ad 5) of the genome.

For producing a plasmid pAd-smmhcLuc, the 1.2 kb BamHI/HindIII smmhc fragment (rabbit “smooth muscle myosin heavy chain” promoter/-1225/-4) is isolated out of the plasmid pRBSMHC-1225βgal (Kallmeier, R. C. et al. (1995) J. Biol. Chem. 270, 30949-30957) and is cloned in the BamHi/HindIII open subclone pBluescript-Luc for the luciferase gene and the subclone p1.2smmhcBluescript-Luc is formed in this way. The BamHi/KpnI smmhc-luciferase fragment of this subclone was then cloned in the BamHI/KpnI interfaces of the plasmid pAd-RSVβgal and the plasmid pAd-smmhcLuc was generated.

[In] the production of plasmid pAd-αmhcLuc containing the “α-myosin heavy chain” promoter (Subramanian, A. et al. (1991) J. Biol. Chem., 266, 24613-24620) was cloned a 1064 bp BamHI/HindIII fragment in the BamHI/HindIII interfaces of the plasmid pBluescript-Luc. A BamHi/KpnI mhc-luciferase fragment thereof was then cloned in the BamHi/KpnI interfaces of the plasmid pAd.RSV-βgal and the plasmid pAD-αmhcLuc was maintained in this way.

### 2. Production of the Recombinant Adenoviruses

The recombined adenoviruses were generated according to the standard methods by homologous recombination among plasmids pAd-Luc, pAd-rsvLuc, pAd-mlcLuc, pAd-smmhcLuc, and pAd-αmhcLuc and genomic DNA of adenoviruses de1324 (Ad5) in 293 cells in vivo (Thimmappaya, B. et al. (1982) Cell 31, 543-551 and Stradtford-Perricaudet, L. D. et al. (1992), cited above, and Graham, F. L. et al. (1977) J. Gen. Virol. 36, 59-74). The recombined adenoviruses possess a deletion in the E3 region and a transgene Luc, RSV-Luc, mlcLuc, pAd-smmhcLuc, and pAd-αmhcLuc substitute the E1 region. On the day before the transfection, 2×10⁶ 293 cells were flattened in a small culture shell. 5 μg of the large C1aI fragment of the genomic DNA of Adde1324 were cotransfected in 293 lines according to the calcium phosphate method, together with 5 μg AatII linearized plasmids pAd-Luc, pAd-RSV-Luc, pAd-mlcLuc, pAd-smmhcLuc, and pAd-αmhcLuc. After coating with soft agar (1% SeaPlaque agarose, 1×MEM, 2% FCS, 100 U/ml penicillin, 0.1 μg/ml streptomycin, 2 mM L-glutamine) and 8-10 days incubation at 37° C. and 5% CO₂, viral plaques were punched out and were clonally multiplied on the 293 cells. The viral DNA of the recombined viruses of 2×10⁶ fully infected 293 cells was isolated and was investigated by hydrolysis by means of a large processing and by means of double cesium chloride density gradient centrifugation (Stradtford-Perricaudet, L. D., 1982, cited above) with respect to the restriction endonucleases as to the correct integration of the transgene. An individual plaque cleaning was undertaken again from the positive viral clones before they multiplied in the 293 cells. Finally, the viruses were dialyzed against TD puffer (137 mM NaCl, 5 mM KCl, 0.7 mM Na₂HPO₄, 0.5 mM CaCl₂, 1 mM MgCl₂, 10% (v/v) glycerin, 25 MM Tris-HCl, pH 7.4), dialyzed, and frozen at −72° C. The “plaque assay” was carried out for determining the titer of the recombined adenoviruses by using 293 cells. All recombined adenoviruses had a titer of approximately 10¹¹ “plaque forming units” (p.f.u.)/ml. The DNA of the viral initial solutions was isolated and investigated by means of an analysis with restriction endonucleases and PCR to the correct integration of the inserts. Furthermore, the viral initial solutions were investigated by means of PCR as to the wild-type Adr, wherein no contamination could be proven (Zang, W. W. et al. (1995) BioTechniques 18, 444-447) in 50 ng of adenoviral DNA.

### 3. Luciferase Detection

For in vitro studies, the cells were gathered 48 hours after infection. The luciferase activity was then determined (Ausubel, F. M. et al. (1989) Current Protocols in Molecular Biology, Greene and Wiley, New York) in protein extracts according to established protocols by means of a Lumat LB 9501 transilluminometer (Bertold, Wildbad). The protein concentration of lysate was determined (BioRad, Munich) according to Bradford (1976). The luciferase activity was calculated in pg luciferase per μg protein (Krougliak, V. & Graham, F. L. (1995) Hum. Gene Ther. 6, 1575-1586 and Franz, W. M. et al. (1993) Circ. Res. 73, 629-638).

For in vivo studies, the rats were decapitated 5 days after the injection. Twelve different tissues (intercostal muscle, heart, thymus, lung, diaphragm, stomach muscle, liver, stomach, spleen, kidneys, quadriceps femoris, brain) were taken and immediately frozen in liquid nitrogen. The tissue samples were then weighed, placed in 200 μl lyse puffer (1% (v/v) triton X-100, 1 mM DTT, 100 mM potassium phosphate pH 7.8), locked in a glass homogenizer, and centrifuged for 15 minutes at 4° C. in a cold centrifuge. The supernatant was used for luciferase detection (Acsadi, G. et al. (1994) Hum. Mol. Gen. 3, 579-584 and Ausubel, F. M. (1989), cited above). The substrate luciferin and ATP were added thereto and the light emission proportional to the luciferase activity was measured photometrically at 560 nm in a transilluminometer. The luciferase activity was given in “relative light units” (RLU)/mg wet tissue weight after removal of the background activity, which was determined for the different tissues in non-infected animals.

### 4. β-Galactosidase Detection

The hearts of neonatal rats were frozen in isopentane cooled in nitrogen and were stored at −70° C. The heart tissue was embedded in O.C.T. (Tissue Tek, Miles, USA) freezing medium and prepared at a 10 μm tissue density with a cryostat (Frigocut 2800 E, Leica). The sections were then fixed for 10 minutes in solution A (PBS, 0.2% (v/v) glutaraldehyde, r mM EDTA, 2 mM MgCl₂), washed 3×10 minutes with solution B (PBS, 0.01% (v/v) sodium-deoxycholate, 0.2% (v/v) nonidet P40, 5 mM EDTA, 2 mM MgCl₂) and colored overnight at 37° C. in solution C (solution B+1 mg/ml X-gal, 5 mM K₃Fe(CN)₆, 5 mM K₄Fe(CN)₆). It was then washed once with solution B and once with distilled water for 10 minutes. A weak countercoloring with hematoxylin and eosin as well as a dehydration and embedding of the samples was carried out according to standard protocols (Gossler, A. & Zachgo, J. (1993) “Gene and Enhancer Trap Screens in ES Cell Chimeras” in Joyner, A. L. (ed.) Gene Targeting, Oxford University Press, 191-225).

### 5. Evidence of Adenoviral DNA with the Aid of PCR Method

Parallel to the luciferase detections according to Example 3, the genomic DNA of the sediments of tissue homogenates of neonatal rats infected with adenoviruses was extracted according to manufacturer specifications with the aid of the QIAamp Tissue Kit (Quiagen Company, Hilden). Two of the animals infected with Ad-Luc, Ad-RSV-Luc and Ad-mlcLuc were investigated as to the tissue distribution of the injected viruses by means of PCR (polymerase chain reaction) to detect adenoviral DNA (Zhang, W. W. (1995) BioTechniques 18, 444-447). For this purpose, 100 ng genomic DNA were used as sample, together with 40 ng oligonucleotides E2B-1 and E2B-2 and 1.25 U Taq polymerase of Promega in a reaction volume of 25 μl. The gel electrophoresis of the specific PCR product yielded an 860 bp band.

The sensitivity of the PCR was determined in previous tests. For this purpose, 100 ng genomic DNA of a non-infected rat were mixed with reduced quantities of Adde1324 DNA and were used as sample in a PCR reaction. To increase the sensitivity of the evidence of PCR, the PCR products were transferred by capillary blot onto a GeneScreenPlus nylon membrane (NEN, Boston, Mass.) and were then detected by Southern hybridization (Ausubel, F. M. et al. (1989), cited above). The PCR amplified adenoviral 860 bp DNA fragment for positive control was used as probe. The PCR product was cleaned of the gel and was radioactively marked by means of “random hexanucleotide prime” with 32 p and was used as sample for the hybridization. The sensitivity of the PCR evidence could be improved in this way by a factor of 10 to 100.

### 6. Infection of Cell Lines (In Vitro)

A10- (smooth muscle cell line of rats), N9c2-((heart myoblast cell line of rats) and HeLa- (human cervix carcinoma cell line) cells were complemented in a “Dulbecco's modified Eagle's medium” (DMEM), 293 cells in MEM, cultivated with 10% fetal calf serum (FCS), 100 U/ml penicillin, 0.1 μg/ml streptomycin, and 2 mM L-glutamine. One day before the infection, 1×10⁵ cells of established cell lines H9c2, A10 and HeLa were flattened in triplicate on “12 well” culture shells. The cells were incubated in 0.2 ml of serum-free medium, which contained the recombined adenoviruses Ad-Luc, Ad-RSVLuc, Ad-mlcLuc, and Ad-smmhcLuc in a “multiplicity of infection” (m.o.i.) of 10 (10 viruses/cell). 2 ml of completed medium were added every 15 minutes after 1 hour incubation at 37° C. under slight oscillation. All the infection experiments were repeated 4 times. The luciferase activities were measured as described above three days after the infections.

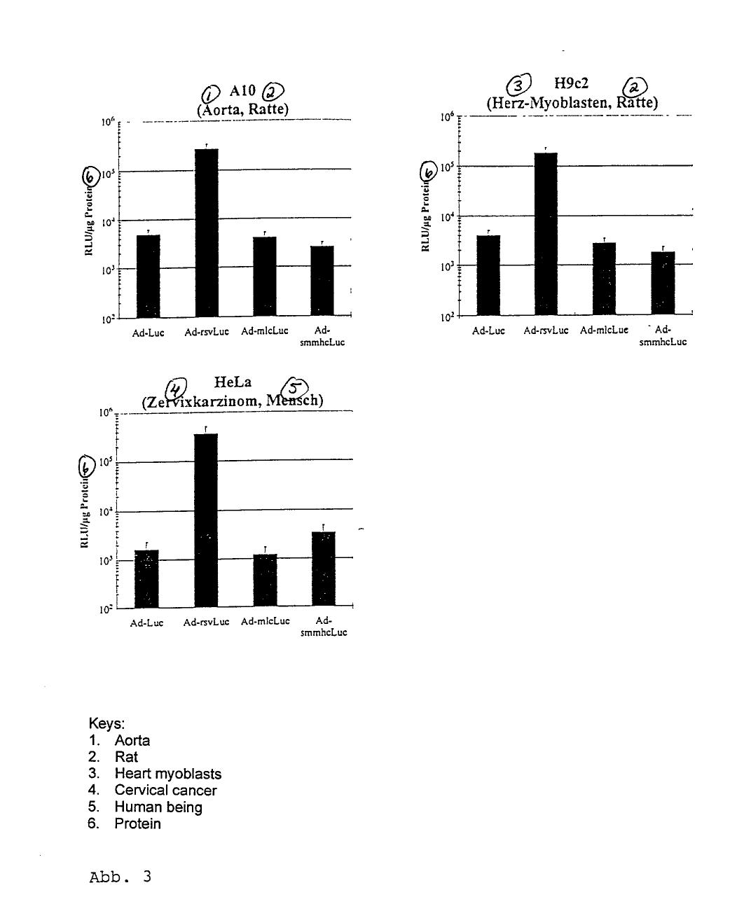

The results of the experiments are shown schematically in FIG. 3. It can be recognized that the luciferase activity of the recombined adenovirus Ad-mlcLuc in all the investigated cell lines is higher than the negative control with the promoter-free adenovirus Ad-Luc. Ad-smmchLuc shows an increased activity in the HeLa cell line and Ad-rsvLuc shows the highest luciferase activity as positive control in all investigated cell lines.

### 7. Infection of Primary Cells in Tissue Culture (In Vitro)

Primary neonatal rat cardiomyocytes of 2 to 3 day old animals were described, prepared, and cultivated by Sen, A. et al. (1988) J. Biol. Chem. 263, 19132-19136. One day before infection, 2×10⁵ cells of freshly prepared neonatal cardiomyocytes were flattened in triplicate on “12 well” culture shells. The cells were incubated in 0.2 ml of serum-free medium, which contained the recombined adenoviruses Ad-Luc, Ad-RSVLuc, Ad-mlcLuc, and Ad-smmhcLuc in a “multiplicity of infection” (m.o.i.) of 10 (10 viruses/cell). 2 ml of completed medium were added every 15 minutes after 1 hour incubation at 37° C. under slight oscillation. All the infection experiments were repeated 4 times. Primary neonatal and adult smooth muscle cells of rats were infected in a similar manner.

The results of the experiments are shown schematically in FIG. 4. It can be recognized that the luciferase activity of the recombined adenovirus Ad-mlcLuc is higher than the negative control with adenovirus Ad-Luc only in the neonatal cardiomyocytes, but is lower than the positive control with the adenovirus Ad-rsvLuc, but 300-900 times higher than in smooth vessel muscle cells. It is also recognized that the luciferase activity of Ad-mlcLuc is 129 times higher than the one of Ad-smmhcLuc. It is concluded that the mlc 2 promoter in neonatal cardiomyocytes is active, while the expected activity of the smmhc promoter in neonatal and adult smooth muscle cells was missing.

### 8. Intercavitary Injection of Recombined Adenoviruses in the Left Heart Cavity of Neonatal Rats

All the injections were carried out on specifically pathogen-free 2-3 day old Spraque Dawley rats (CRWiga, Sulzfeld). Before injection, the neonatal rats were narcotized by 3-5 minute inhalation with methoxyflurane (Metofane, Jannssen Inc.). 2×10⁹ “plaque forming units” (p.f.u.) of the recombined adenoviruses Ad-Luc, Ad-rsvLuc, and Ad-mlcLuc were injected in a volume of 20 μl by means of a tuberculin syringe (27.5 gauge). The injection was carried out by direct puncture of the heart cavity through the lateral rib cage in the 4th intercostal space. It was insured that the needle tip was positioned intracavitarily by means of aspiration of heart blood. A slow injection of viruses (20 μl/min) was obtained by means of a tip of a tuberculin syringe. The injection of recombined adenoviruses in quadriceps femoris was carried out correspondingly.

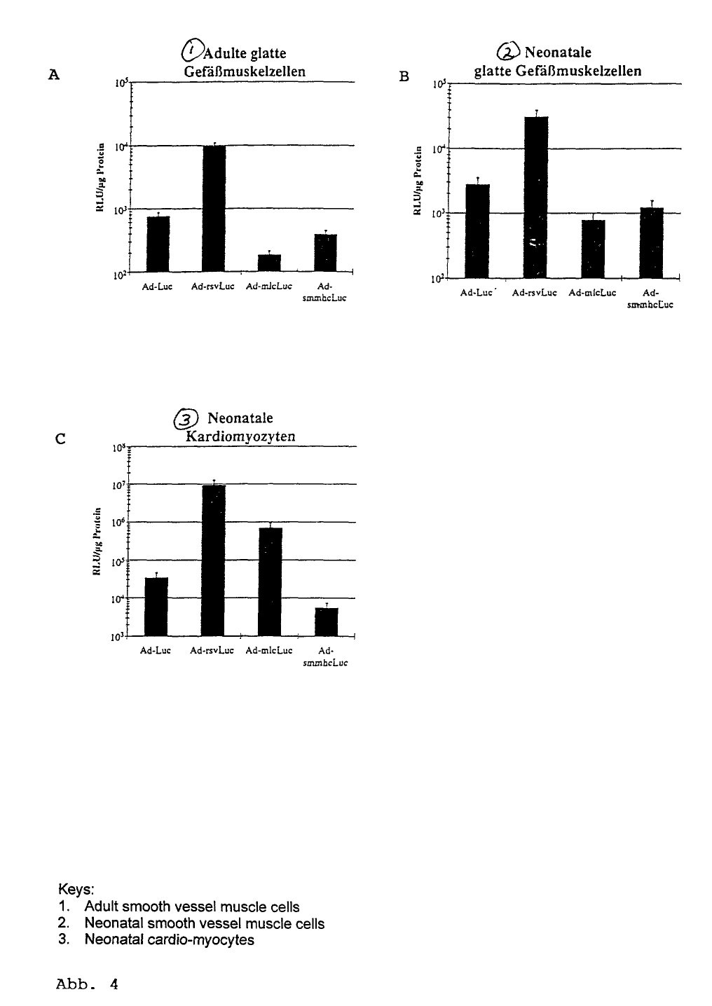

The luciferase activity in twelve different tissues (intercostal muscle, heart, thymus, lung, diaphragm, stomach muscle, liver, stomach, spleen, kidney, brain, and quadriceps femoris) was determined five days after the injection. The determined luciferase activity in RLU/mg tissue is summarized in FIG. 5. The adenovirus Ad-mlcLuc, which carries the heart muscle-specific mlc-2v promoter, shows a luciferase activity that remains limited to the heart muscle (FIG. 5c). The injection of positive control Ad-rsvLuc showed the highest luciferase activity in the intercostal muscle, in the heart, and a strong luciferase activity in the lungs, thymus, and diaphragm (FIG. 5). A lower luciferase activity in the intercostal muscle, heart, thymus, and diaphragm was measured in the Ad-Luc injected animal (FIG. 5a). The Ad-mlcLuc-induced luciferase activity in the heart was 17 times higher than with Ad-Luc, while in all other tissues, the luciferase activity of Ad-Luc and Ad-mlcLuc were comparatively strong. In this way, it was shown that Ad-mlcLuc is specifically active in the heart.

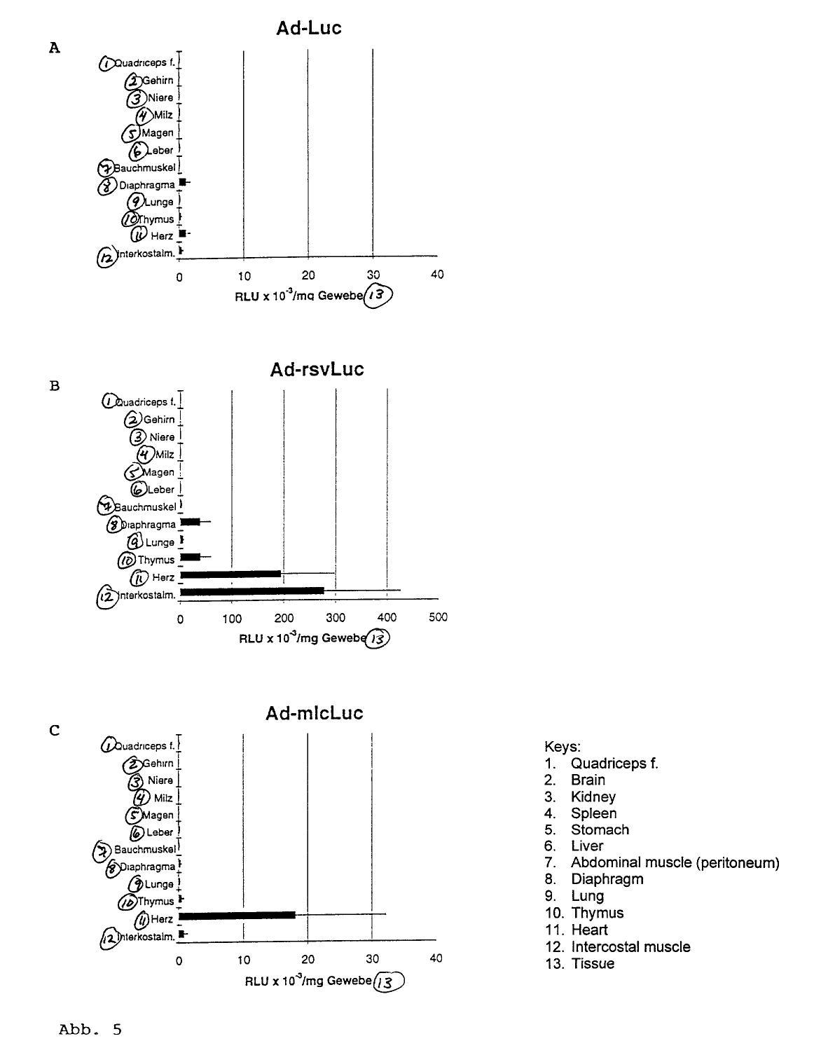

The distribution of infected heart muscle cells after injection of adenoviruses into the heart cavity of neonatal rats was tested additionally in previous experiments by means of an injection of recombined adenovirus Ad-rsvβgal. The recombined adenovirus Ad-rsvβgal expressed the β-galactosidase as report gene under control of the “Rous sarcoma virus” (rsv) promoter. The section of the animal and the expression of the β-galactosidase were determined five days after injection after coloring of the transgene. In the histologic sections, the infected cells are detected by their blue coloration. Approximately half of the myocardial β-galactosidase activity was shown in the region of the injection site in the heart cavity. Along the channel of the injection needle, there was β-galactosidase activity in almost all the cardiomyocytes (FIG. 6a), whereas in the rest of the myocardium the quantity of infected cardiomyocytes was lower (FIG. 6b).

### 9. Injection of Recombined Adenoviruses in the Upper Thigh Muscle of Neonatal Rats

For investigating the activity of the mlc-2v promoter in the skeleton muscle, 20 μl with 2×10⁹ “plaque forming units” (p.f.u.) of three recombined adenoviruses Ad-Luc, Ad-rsvLuc, and Ad-mlcLuc were injected in the right upper thigh quadriceps femoris of neonatal rats. The luciferase activity was determined five days after injection. Ad-Luc and Ad-mlcLuc showed comparatively low luciferase activities (RLU/mg tissue) in the injected thigh, while Ad-rsvLuc was very highly active (Table 1). The luciferase activity obtained by means of Ad-mlcLuc amounted to 0.05% of the luciferase activity of Ad-mlcLuc. This data shows that Ad-mlcLuc is not active in the skeleton muscle and confirms the heart muscle-specific gene expression by means of recombined adenovirus Ad-mlcLuc.

TABLE 1

| | Ad-Luc | Ad-rsvLuc | Ad-mlcLuc |
|---|---|---|---|
| RLU × 10⁻³/mg | 3.4 +/− 1.2 | 5670 +/− 3239 | 2.8 +/− 1.8 |

### 10. Evidence of Adenoviral DNA in Tissues after Injection of Recombined Adenoviruses into the Heart Cavity

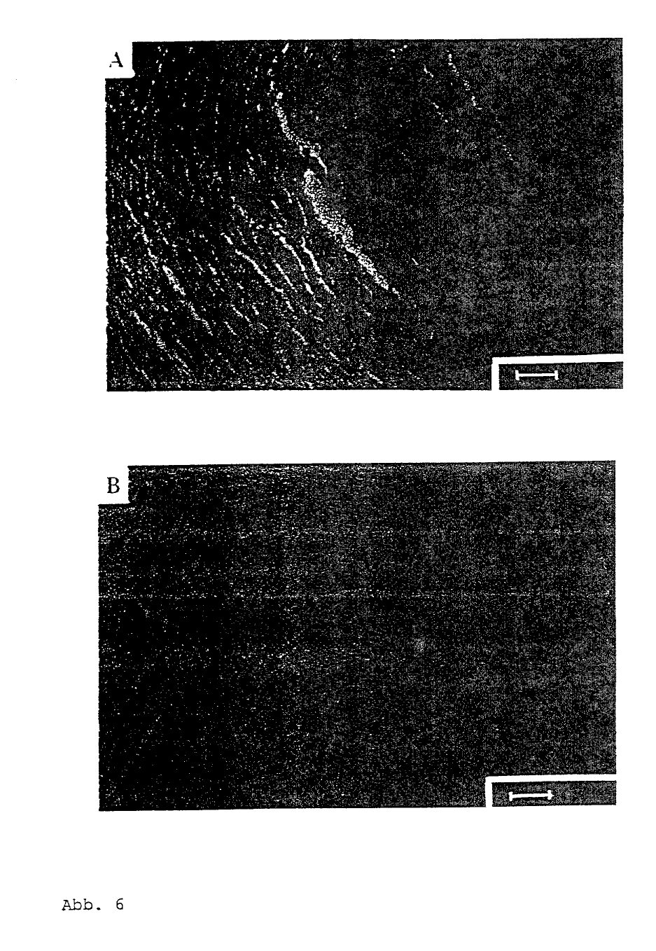

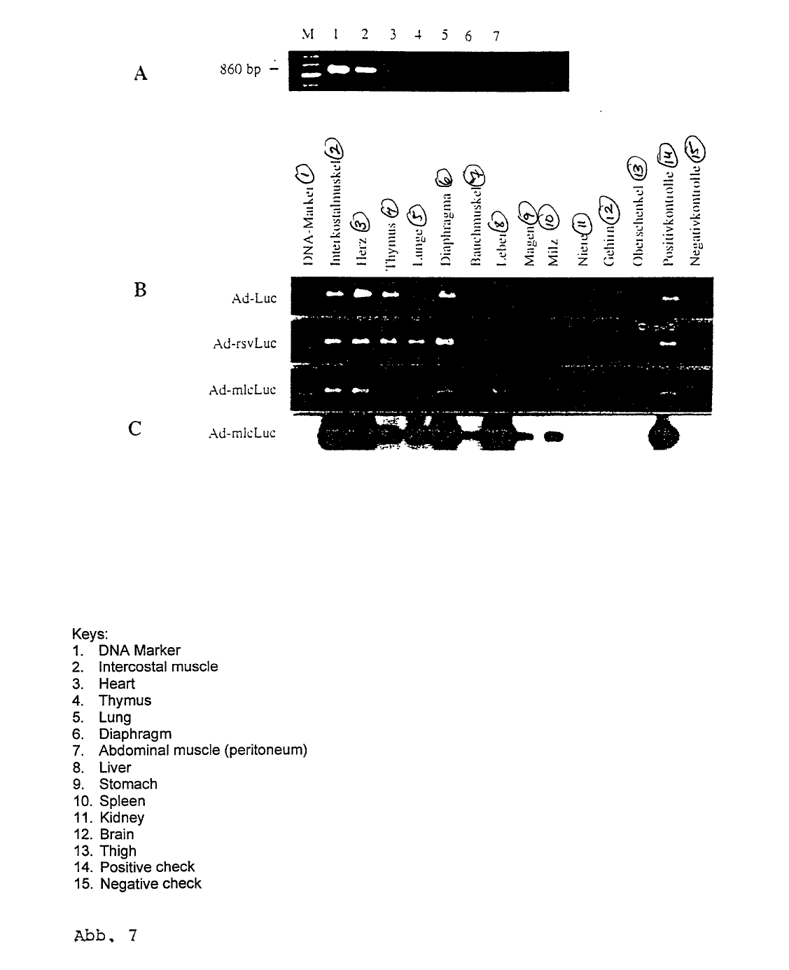

To determine the extent of the infection of non-cardiac tissue after injection of recombined adenoviruses into the heart cavity, the genomic DNA of 12 tissues (intercostal muscle, heart, thymus, lung, diaphragm, stomach muscle, liver, stomach, spleen, kidney, brain, and quadriceps femoris) was isolated and investigated as to the presence of adenoviral DNA in those tissues by means of PCR. The tissues were tested by twos in animals infected with Ad-Luc, Ad-rsvLuc, and Ad-mlcLuc. The sensitivity of the evidence of adenoviral DNA was determined in previous experiments in that 100 ng genomic DNA of non-infected rats were mixed with reduced quantities of adenoviral DNA Ade11324 (from 10 pg to 0.1 fg) and were then investigated. It was shown thereby that 10 fg of the adenoviral DNA Adde1324 could be demonstrated in 100 ng of non-infected animals. This corresponds to 0.017 adenoviral genomes per cell (FIG. 7A). In animals infected with adenovirus, the viral DNA was demonstrated regularly in the intercostal muscle, heart, thymus, lung, diaphragm, and liver (FIG. 4B). To increase the sensitivity of the evidence of the adenoviral DNA, the PCR products were carried over a nylon membrane and were demonstrated by means of Southern blot hybridization. This showed that the adenoviral DNA can also be detected in lower quantities in the other tissues with fewer differences between the individual animals. FIG. 4C shows a representative Southern blot for an Ad-mlcLuc-injected animal.

The described experiments show that the gene expression of the recombined adenovirus Ad-mlcLuc can be attributed to the heart muscle-specific mlc-2v promoter and not to the locally increased virus concentration.

### 11. Comparison of the Specific Activity of the mlc Promoter and the αmhc Promoter

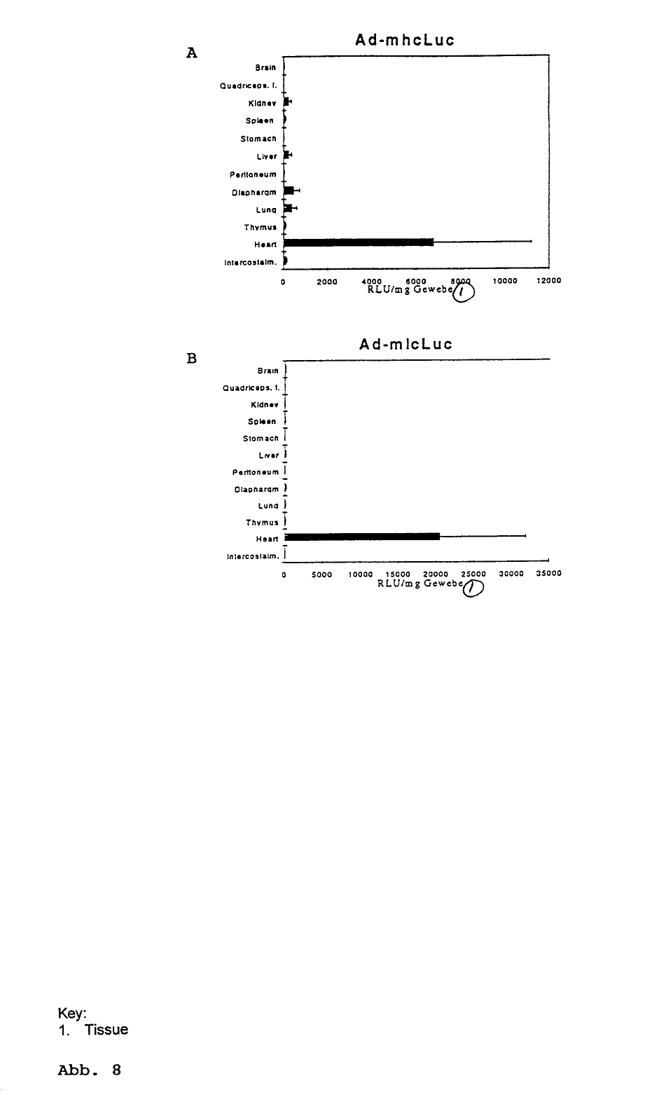

The highest luciferase activity in the heart could be detected for both adenoviruses after intracavitary injection of approx. 2×10⁹ “plaque forming units” of recombined adenoviruses Ad-αmhcLuc (FIG. 8A) and Ad-mlcLuc (FIG. 8B) into the left main cavity of neonatal rats. However, the recombined adenovirus Ad-mlcLuc was 3-4 times more active in the heart than Ad-mhcLuc. The recombined adenovirus Ad-mhcLuc was also more active in the kidney, spleen, liver, diaphragm, lung, and in the intercostal muscle than Ad-mlcLuc. From this it follows that the mlc-2 promoter limits the expression of the luciferase considerably more to the heart in the adenoviral vector system than the αmhc promoter and also that the mlc-2 promoter is 3-4 times more active in the heart than the αmhc promoter.

### 12. Evidence of Ventricle-Specific Expression

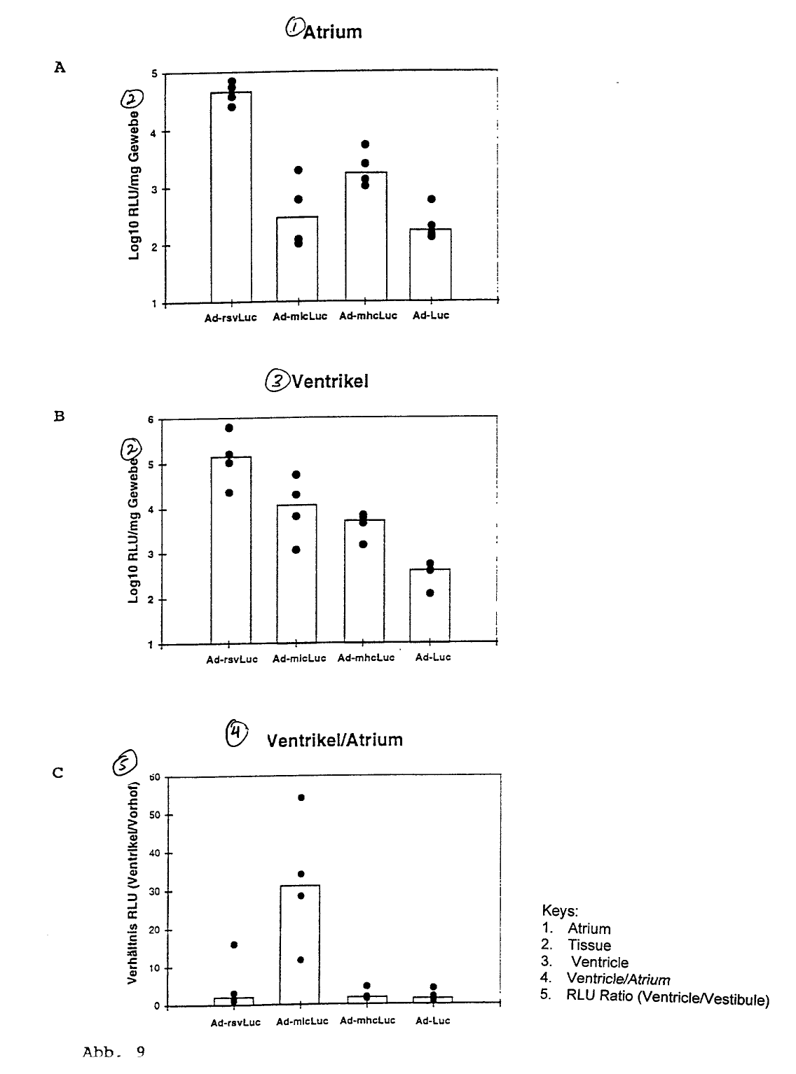

2×10⁹ “plaque forming units” of the recombined adenoviruses Ad-rsvLuc, Ad-mlcLuc, Ad-mhcLuc, and Ad-Luc were injected in a volume of 20-40 μl into the left ventricle of neonatal rats. This tissue was analyzed five days after injection. In four independent experiments, it was shown, that only one gene expression limited to the ventricle could be measured for the recombined adenovirus Ad-mlcLuc (FIG. 9). The relationship of the luciferase activity of Ad-mlcLuc in the ventricle with respect to the atrium amounted to approx. 30, while for all other viruses it amounted to 1-2 times more (FIG. 9C).

1. A gene therapeutic nucleic acid working model containing a regulatory nucleic acid sequence of 5′ end of myosin light chain 2 gene (MLC 2) of the heart that is functionally connected with the nucleic acid, which is encoded for a therapeutically effective gene product, for an antisense nucleic acid, or for a ribosome.

2. A nucleic acid working model according to claim 1, characterized in that the named regulatory nucleic acid sequence comes from the hearts of mammals, particularly humans or rodents, mainly from rats.

3. A nucleic acid working model according to claim 1 or 2, characterized in that the named regulatory nucleic acid sequence comprises the nucleic acids of positions from approximately +18 to −19 up to approximately −800, above all from +18 to −19 up to approximately −1600, and especially from approximately +18 to −19 up to approximately −1800, above all from approximately +18 to −19 up to approximately −2100 or from approximately +18 to −19 up to approximately −2700 with respect to the transcription starting point of the myosin light chain 2 gene (MLC 2) of the heart.

4. A nucleic acid working model according to one of claims 1 to 3, characterized in that the named regulatory nucleic acid sequence comprises the HF 1a element, the HF 1b element, the MLE1 element, and the HF 3 element.

5. A nucleic acid working model according to claim 4, characterized in that the named regulatory nucleic acid sequence also comprises the E box element and/or the HF 2 element.

6. A nucleic acid working model according to claim 4 or 5, characterized in that the named regulatory nucleic acid sequence also comprises the CSS sequence.

7. A nucleic acid working model according to one of claims 1 to 6, characterized in that the nucleic acid sequence is a DNA or RNA sequence, preferably a DNA sequence.

8. A nucleic acid working model according to claim 7, characterized in that the named DNA or RNA sequence is contained in a virus vector.

9. A nucleic acid working model according to claim 8, characterized in that the named DNA sequence sis contained in an adenovirus vector or adeno-associated virus vector, preferably in an adenovirus vector.

10. A nucleic acid working model according to claim 9, characterized in that the named adenovirus vector is a replication deficient adenovirus vector.

11. A nucleic acid working model according to claim 9, characterized in that the named adeno-associated virus vector consists exclusively of two inverted terminal repetition sequences (ITR).

12. A nucleic acid working model according to one of claims 1 to 11, characterized in that the therapeutic gene product is selected from a dystrophin, β adrenergic receptor, or nitrogen monoxide synthesis.

13. A nucleic acid working model according to one of claims 1 to 11, characterized in that the nucleic acid, which is encoded for a therapeutically effective gene product, contains one or several non-encoding sequences and/or one polyA sequence.

14. A process for producing a nucleic acid working model according to one of claims 1-13, characterized in that the named regulatory nucleic acid sequence is functionally connected with a nucleic acid, which encodes for a therapeutically effective gene product, for an antisense nucleic acid, or for ribosome.

15. A process according to claim 14, characterized in that the named nucleic acid sequence is cloned additionally in virus vector according to one of claims 8-11 and/or complexed by means of liposomes.

16. An application of a nucleic acid working model according to one of claims 1-13 for producing a medication for gene therapeutic treatment of heart disease.

17. An application according to claim 16, characterized in that the heart disease is a heart insufficiency, dilative or hypertrophic cardiomyopathy, dystrophinopathy, vessel disorder, high blood pressure, atherosclerosis, stenosis, and/or restenosis of the blood vessels.

18. An application according to one of claims 16 or 17, characterized in that the named medication acts essentially on the heart cavity.

19. Medication containing a nucleic acid working model according to one of claims 1-13 and if necessary a pharmaceutically approved carrier.
